(19) United States
(12) Patent Application Publication
(10) Pub. No.: US 2009/0056727 A1
(43) Pub. Date: Mar. 5, 2009

(54) THUMB- AND FINGER-SUCKING INHIBITION GARMENT AND ASSOCIATED METHODS

(76) Inventor: Carolyn Hill, Windermere, FL (US)

Correspondence Address: James H. Beusse;Beusse Wolter Sanks Mora & Maire, P.A., Suite 2500, 390 North Orange Avenue, Orlando, FL, 32801, US

(21) Appl. No.: 12/201,258

(22) Filed: Aug. 29, 2008

Related U.S. Application Data
(60) Provisional application No. 60/969,284, filed on Aug. 31, 2007.

(51) Int. Cl. A61F 5/37; A41D 11/00; A41D 10/00

(52) U.S. Cl. 128/880; 2/80; 2/83; 2/114

(57) ABSTRACT

A garment for inhibiting thumb and finger sucking includes pajamas having sleeves that cover the hands with a puppet having a likeness of a creature. The child is told a story that has the creature as a character therein. The child is encouraged to participate in the story-telling with the use of the puppet, with the child acting out the part played by the creature in the story. Such an activity engages a child by being part of the action of the story and distracts the child from thumb and or finger sucking. The child is encouraged to wear the garment during the night so that the puppet/creature remains at the end of the sleeves in covering relation to the hands during the night, thereby deterring and inhibiting the child from thumb and or finger sucking. Another element can include positive reinforcement in the form of a reward system.

### SPECIFIC DATA RELATED TO THE INVENTION

This application claims benefit of U.S. Provisional Application No. 60/60/969,284, filed Aug. 31, 2007.

### FIELD OF THE INVENTION

The present invention is directed to garments and methods for inhibiting thumb and finger sucking in small children. This application includes an appendix that details the use of the garment and the disclosure of which is considered an integral part of the invention.

### BACKGROUND OF THE INVENTION

Garments are known for use in attempting to inhibit a child from the habit of thumb and finger sucking with a long-sleeved garment having a mitten-type puppet/character at the end of the sleeves.

However, owing to known problems with such previously known garments, it would be desirable to provide an improved garment and method for inhibiting thumb and finger sucking in small children.

### SUMMARY OF THE INVENTION

The present invention is directed to a garment and associated methods for inhibiting thumb and finger sucking in small children. The method comprises the step of having the child wear a garment that contains sleeves that cover the hands with an element comprising a likeness of a creature in the form of a puppet. Such a creature can comprise, but is not intended to be limited to, a person, an animal, or an imaginary creature or animated character. The method is further delineated in the attached appendix. The next step is to tell the child a story that has the creature as a character in the story. The child is then encouraged to participate in the storytelling with the use of the puppet having the creature's likeness. The child can act out the part played by the creature in the story. Such an activity engages a child by being part of the action described in the story and thereby distracts the child from a thumb- and or finger-sucking activity. The child is then encouraged to wear the garment during the night so that the puppet/creature remains at the end of the sleeves in covering relation to the hands during the night, thereby deterring and inhibiting the child from thumb and or finger sucking.

After at least one night of the child's wearing the garment, the third step is applied, which can include positive reinforcement in the form of a reward system. The method garment and method are intended to deter or inhibit the child from the habit of thumb and or finger sucking for an extended period of time.

### BRIEF DESCRIPTION OF THE DRAWING

FIG. 1 is a front view of a garment of the present invention for the female embodiment; and

FIG. 2 is a front view of a garment of the present invention for the male embodiment.

### DETAILED DESCRIPTION OF THE PREFERRED EMBODIMENTS

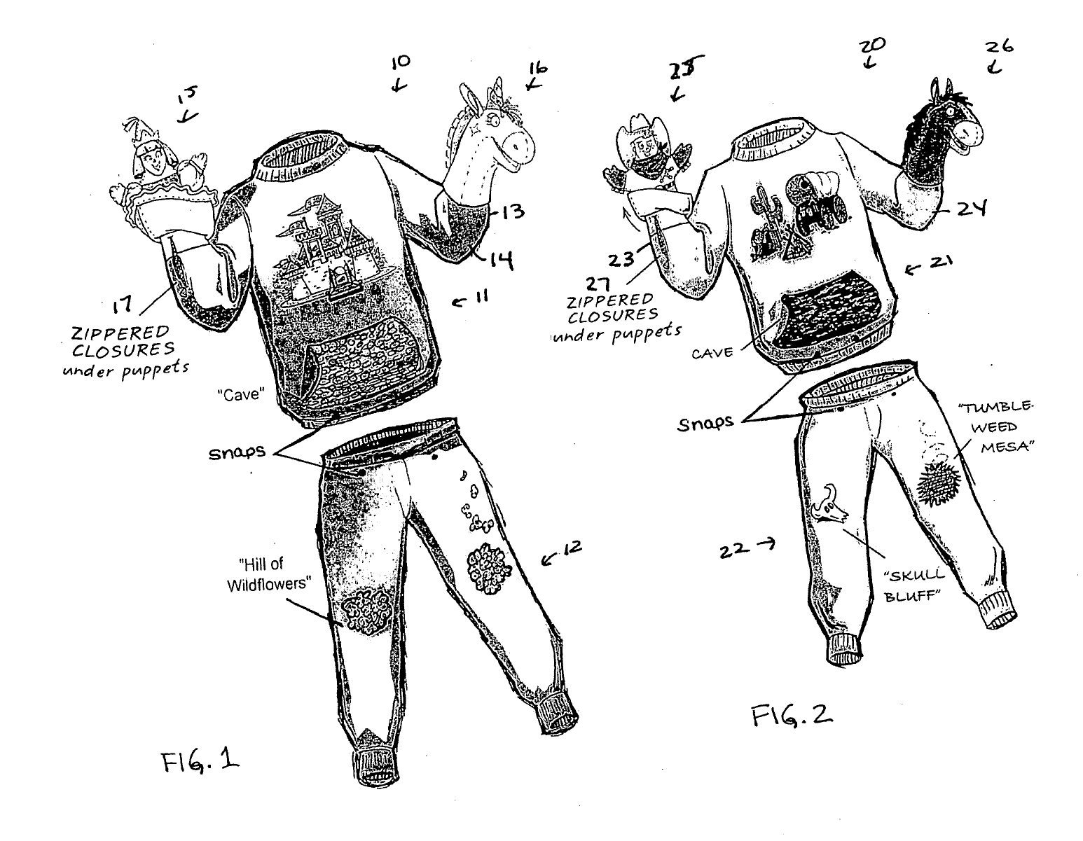

A detailed description of a preferred embodiment of the present invention will now be provided with reference to FIGS. 1 and 2, which represent garment representing substantially the same characteristics and function for females and males, respectively. The present invention is directed to a garment for inhibiting finger and thumb sucking, and is also directed to a method for inhibiting thumb and finger sucking in small children, and a method of making a garment for the purpose of inhibiting finger and thumb sucking.

The garment 10,20 (FIGS. 1 and 2) can comprise a pajama base such as known in the art. Such a garment 10,20 may comprise two-piece pajamas, having fastening elements such as zippers or snaps to connect the upper 11,21 and lower 12,22 sections, in order to prevent the child from taking the pajamas off by him/herself, or may comprise a one-piece garment. The garment 10,20 can also comprise any other long-sleeved garment as a shirt, sweatshirt, sweater, etc., such as known in the art, and does not require the pants section.

Affixed to at least one of the ends 13,23 of the sleeves 14,24 is affixed a character/puppet-type element 15,16;25,26 that is structured to cover the child's thumb and/or one or more fingers. In the embodiment illustrated herein, the character/puppet-type device 15,16;25,26 is illustrated in covering relation to the entire hand, although this is not intended as a limitation.

In a particular embodiment, a zipper-like fastener 17,27 can be attached to the sleeve 14,24 to allow the removal of child's hand from character/puppet at the discretion of the guardian. In alternate embodiments, the character/puppet-type device can be affixed to and removed from the sleeve end 13,23 by other types of fasteners as known in the art, and may be alternately affixable to other garments of the child. The creature embodied in the mitten 15,16;25,26 can comprise, but is not intended to be limited to, a person, an animal, or an imaginary creature or animated character. In a particular embodiment, the mitten 15,16;25,26 can comprise a puppet-type device having one or more manipulable elements such as known in the art of puppet construction. Illustrated in FIGS. 1 and 2, for example, are movable head and arms in the princess mitten 15, a movable mouth in the horse mittens 16,26, and movable head and arms in the cowboy mitten 25.

In a preferred embodiment, the garment 10,20 is marketed with story material, which may be in the form of a book or booklet, or other electronically readable medium such as a tape, CD, or other electronically readable medium such as is known in the art or may be developed in the future. The story, or a series of stories, may also be marketed as being downloadable from the Internet, thus providing an additional revenue stream.

In a method of using the garment 10,20 of the present invention, the child's hand is covered with an element comprising a likeness of a creature, such as the mittens 15,16;25,26 described above, although this is not intended as a limitation.

The guardian then tells or reads the child the story that has the creature(s) therein. The story can be played from a reading medium described above. The child is encouraged to participate in the story-telling step with the use of the puppets, for example, by acting out the part played by the creature in the story. Such an activity engages the child, thereby distracting the child from a thumb-and/or finger-sucking activity. It has been found that the use of a garment such as illustrated herein, combined with a story, can alter the child's habitual behavior in approximately three weeks, although this is not intended as a limitation, and other time spans may be necessary for different children.

In another embodiment, an additional step comprises the use of positive reinforcement in the form of a reward system. Such a reward system can take the form of charts with reward stickers or small prizes for stimulating and motivating the child in a positive way to continue wearing the garment.

The garment/story/reward combination may be used at bedtime or nap time, and may even be used during the day if the thumb and or finger sucking is an issue at other times. After the first night of wearing the garment, the reward system can be established. Further details of the method are included in the attached appendix.

Although the invention has been described relative to specific embodiments thereof, there are numerous variations and modifications that will be readily apparent to those skilled in the art in the light of above teachings. It is therefore to be understood that the invention may be practiced other than as specifically described.

What is claimed is:

1. A system for inhibiting thumb and finger sucking in a child comprising:
a garment in the form of at least a top portion of a pajama having sleeves that terminate in closed ends in the likeness of a puppet type character; and
a book having a story that coordinates with the character on the sleeve ends, the story being structured to require movement of the character.

2. The system of claim 1 and including a reward system associated with the character to encourage use of the character.

3. A garment for inhibiting thumb and finger sucking in a child comprising a pajama top for fitting about an upper body portion of a child and having sleeves extending from the top to the hands of the child, the improvement comprising a mitten enclosing each distal end of the sleeves, the mitten being configured as puppet in the form of a character or creature, each sleeve including a closable slit near the mitten to enable the child's hands to be exposed from the sleeve.
